(19) United States
(12) Patent Application Publication
(10) Pub. No.: US 2026/0345053 A1
(43) Pub. Date: Nov. 5, 2026

(54) System for Multi-Task Estimation of Physiological Parameters Based on Single-Lead Electrocardiogram Signals

(71) Applicant: Feng Chia University, Taichung City (TW)

(72) Inventor: Yue-Der Lin, Taichung City (TW)

(21) Appl. No.: 19/659,241

(22) Filed: Apr. 27, 2026

(30) Foreign Application Priority Data
May 5, 2025 (TW) 114116816

(51) Int. Cl. A61B 5/346; A61B 5/00; A61B 5/28; A61B 5/327; A61B 5/332

(52) U.S. Cl. CPC A61B 5/346; A61B 5/0006; A61B 5/0022; A61B 5/28; A61B 5/327; A61B 5/332; A61B 5/7267

(57) ABSTRACT

Cardiovascular diseases and diabetes pose significant threats to human health. Continuous monitoring of physiological parameters, including blood pressure, baroreflex sensitivity (BRS), and cardiac contractility, is critical for effective disease management. The present invention provides a multitask pipeline neural network system that utilizes a single-lead electrocardiogram (ECG), particularly a lead I ECG signal obtained from a wearable device, as an input to estimate multiple physiological parameters on a beat-to-beat basis. The system is applicable to cuffless continuous blood pressure estimation, pain assessment, bedside monitoring, and diabetes screening. Furthermore, the invention can be extended to evaluate aging indices, brain function, cardiac function, autonomic nervous system (ANS) activity, and overall cardiovascular system performance. The invention provides a scalable and market-competitive solution for home healthcare and medical industries, offering high industrial applicability and commercial value.

### FIELD OF INVENTION

The present invention relates to the field of health monitoring and intelligent medical systems, and more particularly to a system for estimating multiple physiological parameters using a single-lead electrocardiogram (ECG) signal, specifically a lead I ECG signal, through a multitask deep learning neural network architecture.

### BACKGROUND OF THE INVENTION

Conventional clinical monitoring of cardiovascular and metabolic diseases relies on multiple instruments to measure different physiological parameters, such as sphygmomanometers, electrocardiographs, and echocardiography systems. These devices are typically expensive, require trained personnel, and are not suitable for long-term or home-based monitoring. For example, systolic and diastolic blood pressure measurements require cuff-based devices, baroreflex sensitivity requires long-term synchronized recording of ECG and blood pressure, and left ventricular ejection fraction requires imaging-based systems. These limitations restrict their use primarily to clinical settings. Furthermore, traditional cuff-based blood pressure measurement methods cannot achieve true beat-by-beat continuous monitoring and cannot derive arterial blood pressure waveforms or related indices that reflect comprehensive cardiovascular function.

Accordingly, there is a need for a system capable of estimating multiple physiological parameters using only a single-lead ECG signal obtained from a wearable device, thereby reducing medical resource consumption and improving the quality of remote healthcare. Hence, it is eager to have a solution that will overcome or substantially ameliorate at least one or more of the deficiencies of a prior art, or to at least provide an alternative solution to the problems. It is to be understood that, if any prior art information is referred to herein, such reference does not constitute an admission that the information forms part of the common general knowledge in the art.

### SUMMARY OF THE INVENTION

To overcome the aforementioned problems, the present invention provides a system for estimating multiple physiological parameters based on a single-lead electrocardiogram signal, comprising:
a data acquisition module configured to acquire a lead I electrocardiogram signal;
a physiological parameter estimation network module configured to receive the lead I electrocardiogram signal; and
a neural network architecture configured to estimate at least one physiological parameter from the lead I electrocardiogram signal on a beat-to-beat basis.

In some embodiments, the system further comprises a signal conversion module configured to convert the lead I electrocardiogram signal into a lead II electrocardiogram signal, wherein the physiological parameter estimation network module receives the lead II electrocardiogram signal, and the neural network architecture estimates the physiological parameter based on the converted signal.

In some embodiments, the system further comprises a sequence-to-sequence neural network model configured to generate an arterial blood pressure waveform from the physiological information and calculate an augmentation index from the waveform. The arterial waveform may correspond to one or more of the aorta, carotid artery, radial artery, brachial artery, and femoral artery.

In some embodiments, the system further comprises an input embedding module configured to preprocess and segment the ECG signal into fixed-length time-series segments and convert the input data into numerical vectors suitable for neural network processing.

In some embodiments, a positional encoding mechanism is applied to encode sequence order information and is input into a transformer encoder module including a multi-head attention mechanism for capturing long-range dependencies in ECG signals.

In some embodiments, a feedforward neural network is provided following the attention mechanism, together with residual connections and normalization.

In some embodiments, a cloud inference module is configured to transmit signals and/or estimated physiological parameters to a remote platform for processing and feedback.

In some embodiments, the data acquisition module is implemented as a wearable device, including a smartwatch, ring, or wristband.

In some embodiments, the physiological parameters include a 12-lead ECG, blood pressure, baroreflex sensitivity, and/or left ventricular ejection fraction.

In accordance, the present invention has the following advantages:

First, compared with conventional technologies, the present invention differs not only in its modular configuration and neural network architecture, but more importantly in that the present invention performs blood pressure estimation on a beat-to-beat basis. Such a feature provides significant practical and clinical value. In particular, the present invention requires only a single-lead electrocardiogram (ECG) signal as an input to simultaneously estimate multiple physiological parameters, including but not limited to blood pressure, baroreflex sensitivity (BRS), and left ventricular ejection fraction (LVEF), thereby substantially simplifying conventional diagnostic workflows that typically require multiple independent medical devices. Furthermore, by integrating a cloud-based computing platform, the system is capable of providing real-time analysis and feedback, thereby improving the timeliness and convenience of health monitoring.

Second, the present invention is applicable to a wide range of medical environments, including home healthcare settings, intensive care units (ICUs), and operating rooms, and is particularly suitable for long-term monitoring and preventive screening of chronic diseases such as diabetes and hypertension. The arterial blood pressure waveform and augmentation index generated by the present invention can serve as important indicators for evaluating arterial stiffness and cardiovascular risk, thereby providing high clinical relevance. Moreover, the present invention can be extended to various applications, including cuffless continuous blood pressure estimation, pain assessment, bedside monitoring, and diabetes screening. The system may also be further applied to the evaluation of aging-related indices, brain function, cardiac function, autonomic nervous system activity, and overall cardiovascular system performance. Accordingly, the present invention provides a technologically competitive solution for home healthcare and medical industries, offering high industrial applicability and added value. In addition, the present invention has the potential to facilitate the implementation of digital twin-based healthcare systems and provides a foundational technology for intelligent medical applications. Many of the attendant features and advantages of the present invention will become better understood with reference to the following detailed description considered in connection with the accompanying figures and drawings.

### BRIEF DESCRIPTION OF THE DRAWINGS

The steps and the technical means adopted by the present invention to achieve the above and other objects can be best understood by referring to the following detailed description of the preferred embodiments and the accompanying drawings.

FIG. 1 is a schematic diagram illustrating an overall system architecture and relationships among modules of the system according to the present invention.

FIG. 2 is a flowchart illustrating method steps of the present invention.

FIG. 3 is a schematic diagram illustrating a multitask pipeline neural network architecture according to the present invention.

FIG. 4 is a schematic diagram illustrating a neural network architecture for estimating systolic blood pressure on a beat-by-beat basis from electrocardiogram signals according to the present invention.

### DETAILED DESCRIPTION OF THE PREFERRED EMBODIMENTS

The present invention will be described in detail below with reference to several preferred embodiments. It should be understood that the accompanying drawings are provided for illustrative purposes only and represent exemplary embodiments of the present invention, and are not intended to limit the scope of the invention. Those having ordinary skill in the art may make various modifications and apply the present invention to other similar embodiments without departing from the spirit and scope of the invention.

As used herein, the terms “system,” “device,” “unit,” and/or “module” are used to distinguish different levels of components, elements, parts, or assemblies. However, these terms are not intended to be limiting, and other terminology may be used interchangeably to achieve the same purpose. Unless explicitly indicated otherwise by the context, the terms “a,” “an,” and “the” are intended to include both singular and plural forms. In general, the terms “comprising” and “including” indicate the presence of stated features, elements, steps, or components, but do not preclude the presence or addition of one or more other features, elements, steps, components, or groups thereof.

Flowcharts are used herein to illustrate operations performed by the system according to embodiments of the present invention. It should be understood that the operations described herein are not necessarily performed in a strict sequential order. Rather, the operations may be performed in reverse order, in parallel, or with additional steps inserted or certain steps omitted.

### Embodiment 1 of System for Multi-Task Estimation of Physiological Parameters Based on Single-Lead ECG Signals

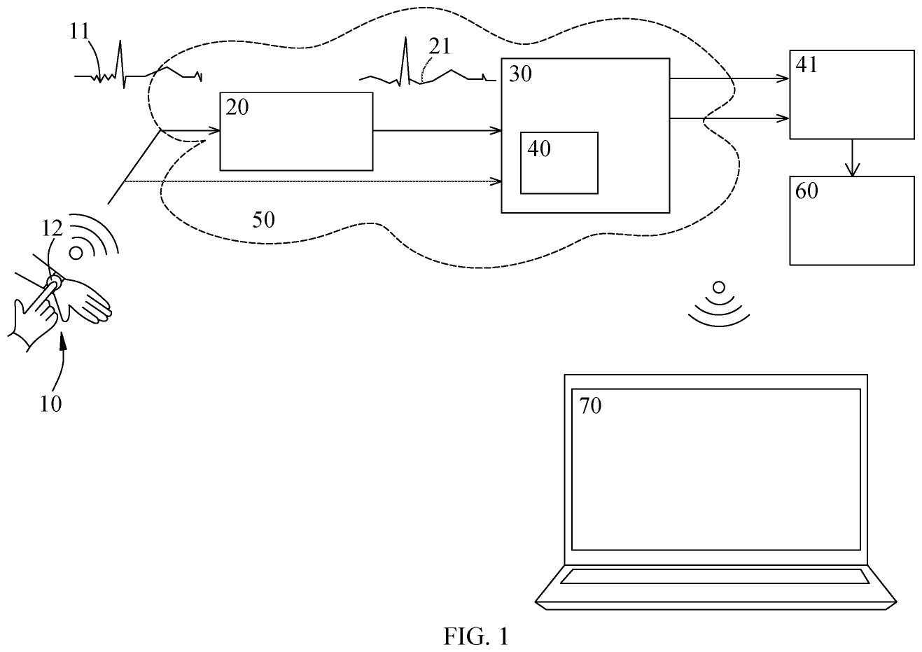

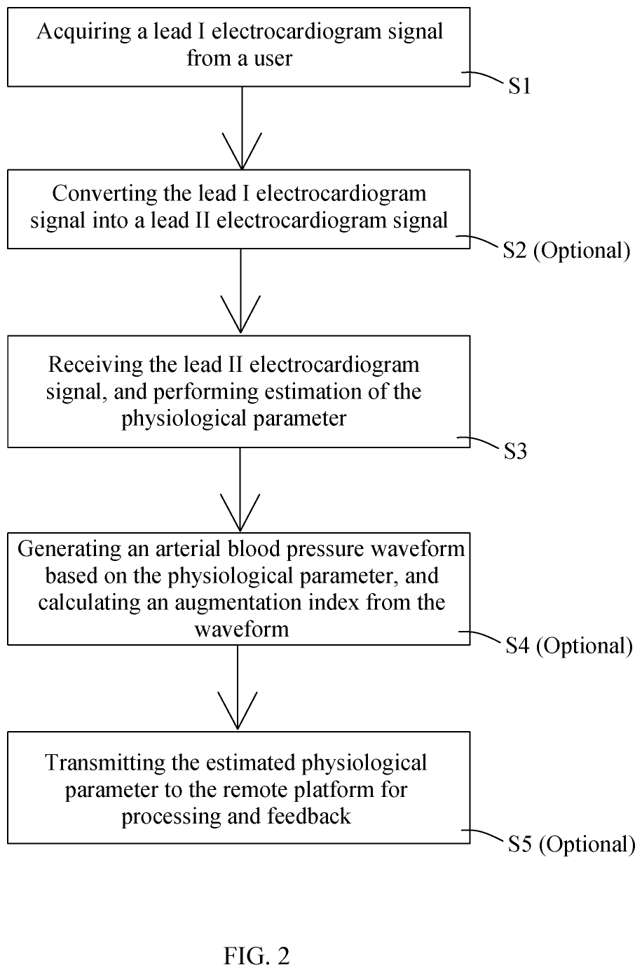

Referring to FIGS. 1-4, which illustrate the overall system architecture and relationships among modules, the system for multi-task estimation of physiological parameters based on a single-lead electrocardiogram signal according to the present invention comprises:
a data acquisition module 10 configured to acquire a lead I (lead one) electrocardiogram (ECG) signal 11;
a signal conversion module 20 configured to convert the lead I electrocardiogram signal 11 into a lead II (lead two) electrocardiogram signal 21;
a physiological parameter estimation network module 30 configured to receive the converted lead II electrocardiogram signal; and
a neural network architecture 40 configured to estimate at least one physiological parameter 41 on a beat-to-beat basis.

In some preferred embodiments, the system further comprises a cloud inference module 50, wherein the signals may be transmitted in real time to a remote platform 70 for processing and feedback.

In some embodiments, the data acquisition module 10 is implemented as a wearable device 12, including but not limited to a smartwatch, a ring, or a wristband, configured to be attached to a user and to sense physiological conditions of the user. In one preferred example, the wearable device is worn on a left wrist of the user, and the lead I electrocardiogram signal 11 is acquired when the user touches a crown portion of the wearable device with a finger of the other hand.

The physiological parameter 41 includes, but is not limited to, a 12-lead electrocardiogram, blood pressure (BP), baroreflex sensitivity (BRS), and/or left ventricular ejection fraction (LVEF). In some embodiments, the blood pressure includes systolic blood pressure (SBP) and/or diastolic blood pressure (DBP).

### Embodiment 2 of System for Multi-Task Estimation of Physiological Parameters Based on Single-Lead ECG Signals

A second preferred embodiment is substantially similar to the first embodiment described above, except that the system further comprises a sequence-to-sequence (Seq2Seq) neural network model 60. The sequence-to-sequence neural network model 60 is configured to generate an arterial blood pressure waveform from the physiological parameter and to calculate an augmentation index based on the waveform. In preferred embodiments, the arterial blood pressure waveform corresponds to one or more arterial locations, including an aorta, a carotid artery, a radial artery, a brachial artery, and/or a femoral artery. The augmentation index represents an index for evaluating arterial stiffness of a user.

Similarly, the arterial blood pressure waveform and the augmentation index may be transmitted to the cloud platform for processing, analysis, and feedback.

### Embodiment 3 of System for Multi-Task Estimation of Physiological Parameters Based on Single-Lead ECG Signals

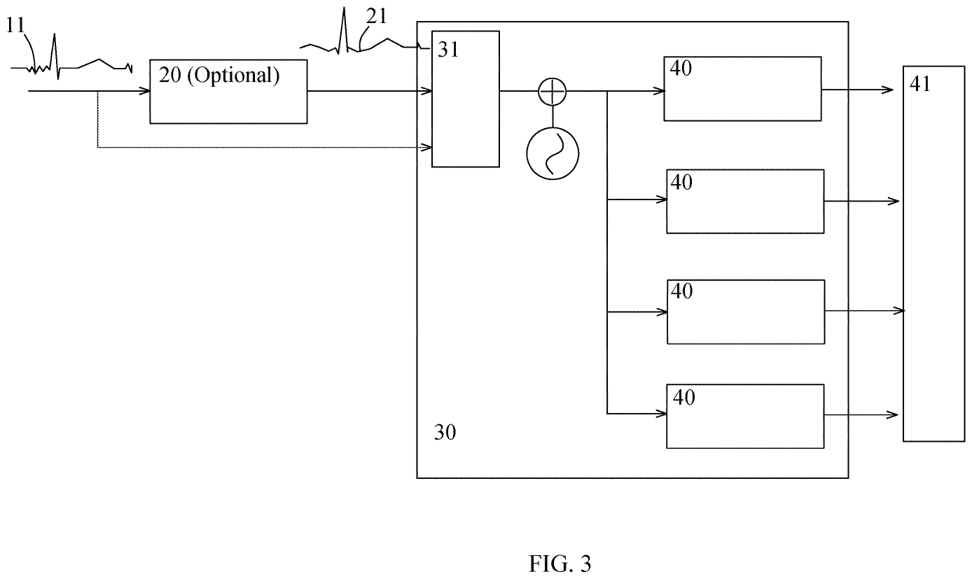

Also shown in FIG. 3, a third preferred embodiment is substantially similar to the first embodiment, except that the system does not include the signal conversion module 20. Instead, the physiological parameter estimation network module 30 directly receives the lead I electrocardiogram signal 11, and the neural network architecture 40 performs estimation of the physiological parameter 41 on a beat-to-beat basis. In order to ensure that the present invention is implemented as a practical technological solution rather than an abstract concept, the system is embodied in a combination of physical hardware components and signal processing modules. The data acquisition module is implemented as a wearable physiological sensing device configured to directly contact a user and acquire bioelectrical signals. The wearable device includes at least one electrode sensor configured to detect electrical cardiac activity and generate a lead I electrocardiogram (ECG) signal. The wearable device further includes an analog front-end (AFE) circuit configured to amplify and filter the acquired ECG signal, and an analog-to-digital converter (ADC) configured to digitize the ECG signal for subsequent processing. The system further comprises a processing unit electrically coupled to the data acquisition module. The processing unit includes at least one microcontroller, digital signal processor (DSP), or application-specific integrated circuit (ASIC), configured to execute signal preprocessing, feature extraction, and neural network inference operations. In some embodiments, the processing unit is integrated within the wearable device. In other embodiments, the processing unit is implemented in an external computing device, including a smartphone, edge computing device, or cloud server. The physiological parameter estimation network module and the neural network architecture are implemented as executable program instructions stored in a non-transitory computer-readable storage medium and executed by the processing unit. The execution of the neural network architecture transforms the acquired ECG signal into quantified physiological parameters, thereby converting raw bioelectrical signals into clinically meaningful outputs. The system further includes a communication module configured to transmit the ECG signal and/or the estimated physiological parameters to a remote platform via a wireless communication protocol, including Bluetooth, Wi-Fi, or cellular communication. The remote platform comprises one or more servers configured to perform additional computation, data storage, and real-time feedback generation. In some embodiments, the system further includes a user interface configured to display the estimated physiological parameters, including blood pressure values, arterial waveforms, and derived indices. The user interface may be implemented on the wearable device, a mobile device, or a remote display system. Accordingly, the present invention provides a specific implementation that integrates physiological signal acquisition hardware, signal conditioning circuits, digital processing units, and neural network-based computation to achieve real-time estimation of physiological parameters. The invention therefore constitutes a practical application of signal processing and machine learning technologies applied to a physical system, and is not merely a mental process or abstract mathematical calculation.

### <Method for Multi-Task Estimation of Physiological Parameters Based on Single-Lead ECG Signals>

The present invention further provides a method for estimating the physiological parameter 41 using the aforementioned system, wherein the method establishes a defined transformation relationship between an input electrocardiogram signal and one or more output physiological parameters, and comprises the following steps:

Step S1: acquiring, by the data acquisition module 10, a lead I electrocardiogram (ECG) signal 11 from a user, wherein the ECG signal represents time-series bioelectrical measurements corresponding to cardiac activity.

Step S2: converting, by the signal conversion module 20, the lead I electrocardiogram signal 11 into a lead II electrocardiogram signal 12 for use in subsequent estimation and labeled dataset preparation, and segmenting the ECG signal into individual heartbeat segments based on detected R-waves. R-wave indices are first detected using an R-wave detection algorithm, and each heartbeat segment is defined by extracting a fixed number of sampling points before and after each R-wave. Specifically, each ECG segment is defined as a window centered at an R-wave, for example including sampling points before and 30 sampling points after the R-wave, thereby forming a segment corresponding to a single cardiac cycle. Each ECG segment is therefore represented as a fixed-length time-series vector and used as an input sample to the neural network. In preferred embodiments, the lead II electrocardiogram signal 12 is segmented based on an R-wave reference point to form a plurality of fixed-length ECG segments, each ECG segment corresponding to a single cardiac cycle. Each ECG segment is defined by a predetermined number of sampling points before and after the R-wave, such that each segment encodes a complete P-QRS-T waveform, wherein a fixed-length segment is extracted before and/or after the R-wave. For example, segment formats such as 20R40 or 35R35 may be used, where the notation indicates the number of sampling points before and after the R-wave. The R-wave corresponds to the most prominent upward deflection within the QRS complex and represents ventricular depolarization. Accordingly, a “20R40” segment includes 20 sampling points preceding the R-wave and 40 sampling points following the R-wave, while a “35R35” segment includes 35 sampling points before and after the R-wave. By centering the ECG segment around the R-wave and extracting a fixed-length segment, the input ECG data can be temporally aligned, thereby capturing a complete cardiac cycle including P-QRS-T waveform features. By centering the ECG segment at the R-wave, the resulting input vectors are temporally aligned across cardiac cycles, thereby ensuring consistency in feature representation and enabling the neural network to learn physiologically meaningful patterns. More preferred embodiment, In conjunction with the ECG segmentation, corresponding blood pressure values are obtained from arterial blood pressure (ABP) waveform signals. For each ECG segment indexed by an R-wave R[i], the corresponding systolic blood pressure (SBP) and diastolic blood pressure (DBP) are defined based on the ABP waveform as follows:

SBP[i] is defined as a maximum value of the ABP signal within an interval from the R-wave R[i] to a subsequent R-wave R[i+1]; and DBP[i] is defined as a minimum value of the ABP signal within a region proximate to the subsequent R-wave R[i+1].

Accordingly, each ECG segment corresponds to one pair of SBP and DBP values derived from the ABP waveform, thereby forming a labeled dataset in which each input ECG segment has a defined physiological output.

Step S3: receiving, by the physiological parameter estimation network module 30, the lead II electrocardiogram signal 12, and performing estimation of the physiological parameter 41 using the neural network architecture 40. Preferably, the physiological parameter estimation is performed on a beat-to-beat basis. In this step, the neural network learns a mapping relationship between ECG waveform features and corresponding blood pressure values, such that each ECG segment corresponding to a cardiac cycle is mapped to a predicted systolic blood pressure and/or diastolic blood pressure value. Accordingly, the estimation is performed on a beat-to-beat basis, wherein each ECG segment produces a corresponding output physiological parameter.

Step S4 (optional): generating, by the sequence-to-sequence neural network model 60, an arterial blood pressure waveform based on the physiological parameter, and calculating an augmentation index from the waveform.

Step S5 (optional): transmitting, by the cloud inference module 50, the estimated physiological parameter 41 to the remote platform 70 for processing and feedback, and optionally transmitting the estimation results back to the wearable device 12 to provide real-time health status feedback.

In preferred embodiments, in Step S3, the physiological parameter estimation network module 30 utilizes a transformer-based neural network architecture, or other neural network architectures not limited thereto, to perform multiple tasks, including blood pressure estimation (systolic blood pressure (SBP) and diastolic blood pressure (DBP)), baroreflex sensitivity (BRS) estimation, left ventricular ejection fraction (LVEF) estimation, arterial waveform generation (via the Seq2Seq model), and augmentation index calculation. Specifically, the lead II electrocardiogram signal 12 is input into an input embedding module 31, wherein the continuous ECG signal is preprocessed and segmented into fixed-length time-series segments. The segmented data are then converted into numerical vectors suitable for neural network processing.

A positional encoding mechanism 32 is applied to encode sequence order information, and the encoded data are then input into a transformer encoder module 33. The transformer encoder module 33 includes a multi-head attention (MHA) mechanism. In one embodiment, the multi-head attention mechanism employs eight attention heads (nhead=8) to extract dependencies among sequence features from multiple perspectives, thereby effectively capturing long-range temporal dependencies in the electrocardiogram signal.

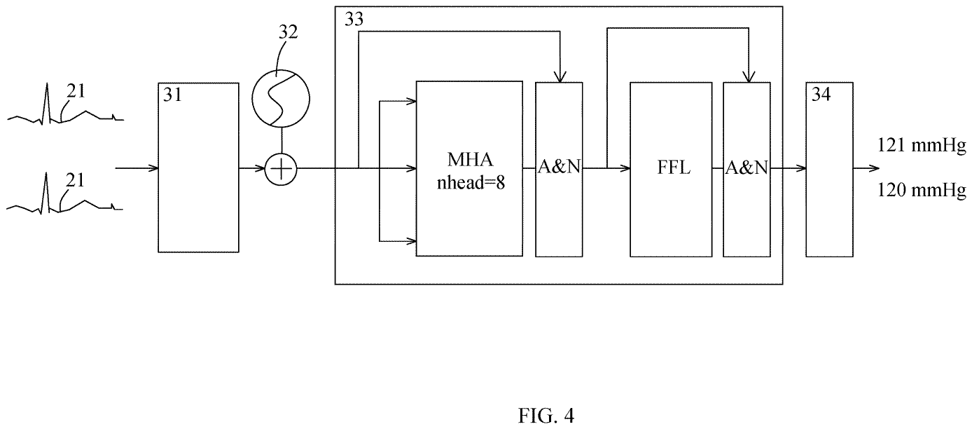

A feedforward layer (FFL) is arranged after the attention mechanism, and is combined with residual connections and a normalization mechanism (Add & Norm) to stabilize training and improve performance. In some embodiments, multiple transformer encoder layers may be stacked to enhance modeling capability. After encoding by the transformer encoder module 33, the resulting sequence features are input into a linear layer 34 or a multilayer perceptron to perform regression prediction, thereby generating predicted physiological parameter values, such as blood pressure values at consecutive time points (e.g., 120 mmHg, 121 mmHg, etc., as illustrated in FIG. 4).

### <Validation Test>

In one implementation, the present invention utilizes data collected from 2,378 subjects, resulting in a total of 2,999,829 lead I electrocardiogram recordings. These recordings are converted into a lead II ECG dataset labeled with corresponding blood pressure values. The signal quality of the dataset is verified and confirmed by clinical professionals. During testing, ECG segments are extracted using the R-wave as a reference point, and fixed-length segments are selected before and after the R-wave as input to the physiological parameter estimation network module 30 and the sequence-to-sequence neural network model 60. In one example, the sampling frequency of the ECG signal is 125 Hz, and a “20R40” segment corresponds to 0.16 seconds (20 sampling points) before the R-wave and 0.32 seconds (40 sampling points) after the R-wave.

Table 1 illustrates the performance of the system and method of the present invention for blood pressure estimation based on electrocardiogram signals. The performance is evaluated according to AAMI and BHS standards, wherein cumulative error, mean error (ME), and standard deviation (STD) are used as evaluation metrics.

TABLE 1

| | Systolic | | | | | | |
|---|---|---|---|---|---|---|---|
| | blood | | | | | | |
| | pressure/ | | | | Mean | Standard | |
| | Diastolic | Cumulative Error | | | Error | Deviation | |
| | blood | < 5 | ≤ 10 | ≤15 | (ME, | (STD, | |
| Group | pressure | mmHg | mmHg | mmHg | (mmHg) | mmHg | Grade |
| Standard | Grade A | 60% | 85% | 95% | NA | NA | — |
| 1 | Grade B | 50% | 75% | 90% | NA | NA | |
| BHS | Grade C | 40% | 65% | 85% | NA | NA | |
| [symbol] 2 | NA | NA | NA | NA | <5 | <8 | |
| AAMI | | | | | | | |
| Embodiment | Systolic | 70.80% | 90.09% | 95.39% | 0.0165 | 7.4002 | A |
| 1 | blood | | | | | | |
| 20R40 | pressure | | | | | | |
| | Diastolic | 88.17% | 97.25%\| | 99.07% | −0.2131 | 3.8001 | A |
| | blood | | | | | | |
| | pressure | | | | | | |
| Embodiment | Systolic | 71.19% | 90.18% | 95.41% | −0.1497 | 7.4092 | A |
| 2 | blood | | | | | | |
| 35R35 | pressure | | | | | | |
| | Diastolic | 88.00% | 97.19% | 99.07% | −0.0095 | 3.8321 | A |
| | blood | | | | | | |
| | pressure | | | | | | |
| Embodiment | Systolic | 70.85% | 90.31% | 95.54% | −0.0889 | 7.4380 | A |
| 3 | blood | | | | | | |
| 40R40 | pressure | | | | | | |
| | Diastolic | 89.19% | 97.38% | 99.12% | −0.0524 | 3.7017 | A |
| | blood | | | | | | |
| | pressure | | | | | | |

As shown in Table 1, the results of Embodiments 1 to 3 of the present invention demonstrate that the proposed system achieves Grade A performance under the BHS standard and also satisfies the AAMI standard for blood pressure estimation.

In some embodiments, numerical values are used to describe quantities of components or properties. It should be understood that such numerical values may be modified by terms such as “about,” “approximately,” or “substantially.” Unless otherwise specified, these terms indicate that the corresponding numerical values may vary within a range of ±20%. Accordingly, in certain embodiments, numerical parameters set forth in the specification and claims are approximations that may vary depending on the desired characteristics of a particular embodiment. In some cases, numerical values should be interpreted in view of the number of significant digits and ordinary rounding techniques. Although numerical ranges and parameters are approximate in nature, they are set forth as precisely as practicable in the context of the invention.

Finally, it should be understood that the embodiments described herein are provided for illustrative purposes only and are not intended to limit the scope of the present invention. Various modifications and variations may be made by those skilled in the art without departing from the spirit and scope of the invention. Accordingly, all such modifications and equivalent arrangements are intended to be included within the scope of the present invention as defined by the appended claims. The above specification, examples, and data provide a complete description of the present disclosure and use of exemplary embodiments. Although various embodiments of the present disclosure have been described above with a certain degree of particularity, or with reference to one or more individual embodiments, those with ordinary skill in the art could make numerous alterations or modifications to the disclosed embodiments without departing from the spirit or scope of this disclosure.

What is claimed is:

1. A system for multi-task estimation of physiological parameters based on a single-lead electrocardiogram signal, comprising:
a data acquisition module comprising at least one electrode sensor configured to be in contact with a user and to detect bioelectrical cardiac signals;
a processing unit comprising at least one processor and a computer-readable storage medium storing program instructions;
a physiological parameter estimation network module implemented by the processor and configured to receive the lead I electrocardiogram signal;
a neural network architecture implemented by the processor and configured to process the lead I electrocardiogram signal and to estimate at least one physiological parameter from the lead I electrocardiogram signal on a beat-to-beat basis; wherein the processing of the lead I electrocardiogram signal by the neural network architecture transforms the bioelectrical cardiac signals into quantified physiological parameters corresponding to cardiac cycles; and
wherein the physiological parameter comprises a 12-lead electrocardiogram, blood pressure, baroreflex sensitivity, and/or left ventricular ejection fraction.

2. The system of claim 1, further comprising a signal conversion module implemented by the processing unit and configured to convert the lead I electrocardiogram signal into a lead II electrocardiogram signal,
wherein the physiological parameter estimation network module executed by the processing unit is configured to receive the lead II electrocardiogram signal, and
wherein the neural network architecture executed by the processing unit is configured to estimate the physiological parameter from the lead II electrocardiogram signal on a beat-to-beat basis.

3. The system of claim 1, further comprising a sequence-to-sequence neural network model implemented by the processing unit and configured to generate an arterial blood pressure waveform from the physiological parameter and to calculate an augmentation index based on the arterial blood pressure waveform.

4. The system of claim 2, further comprising a sequence-to-sequence neural network model implemented by the processing unit and configured to generate an arterial blood pressure waveform from the physiological parameter and to calculate an augmentation index based on the arterial blood pressure waveform.

5. The system of claim 2, further comprising an input embedding module implemented by the processing unit and configured to preprocess and segment the lead II electrocardiogram signal into fixed-length time-series segments, and to convert the lead II electrocardiogram signal into numerical vectors suitable for neural network processing.

6. The system of claim 5, further comprising a positional encoding mechanism and a transformer encoder module implemented by the processing unit, wherein the transformer encoder module includes a multi-head attention mechanism configured to extract dependencies among sequence features and capture long-range temporal relationships in the electrocardiogram signal.

7. The system of claim 6, further comprising a feedforward neural network implemented by the processing unit and arranged after the multi-head attention mechanism, and further including residual connections and a normalization mechanism.

8. The system of claim 2, further comprising a cloud inference module implemented by the processing unit and a communication module configured to transmit the lead I electrocardiogram signal, the lead II electrocardiogram signal, and/or the physiological parameter in real time to a remote platform via a wireless communication protocol for processing and feedback.

9. The system of claim 1, wherein the data acquisition module is implemented as a wearable device configured to be attached to a user and to sense a physiological state of the user, the wearable device including a smartwatch, a ring, or a wristband.

10. The system of claim 2, wherein the data acquisition module is implemented as a wearable device configured to be attached to a user and to sense a physiological state of the user, the wearable device including a smartwatch, a ring, or a wristband.

11. The system of claim 1, wherein the blood pressure comprises systolic blood pressure and/or diastolic blood pressure.

12. The system of claim 3, wherein the arterial blood pressure waveform corresponds to one or more of an aorta, a carotid artery, a radial artery, a brachial artery, and a femoral artery, and wherein the augmentation index represents an arterial stiffness index of a user.
